(19) United States
(12) Patent Application Publication
(10) Pub. No.: US 2023/0131256 A1
(43) Pub. Date: Apr. 27, 2023

(54) STENT

(71) Applicant: optimed medizinische Instrumente GmbH, Ettlingen (DE)

(72) Inventors: Thomas WILLE, Senftenberg (DE); Thilo WACK, Tholey (DE); Fabian SCHMIDT, Karlsruhe (DE); Achim ZIPSE, Baden-Baden (DE)

(21) Appl. No.: 17/970,750

(22) Filed: Oct. 21, 2022

(30) Foreign Application Priority Data
Oct. 22, 2021 (DE) 10 2021 127 510.5

(51) Int. Cl. A61F 2/89

(52) U.S. Cl. CPC A61F 2/89; A61F 2210/0004; A61F 2220/0083; A61F 2230/0069; A61F 2240/001

(57) ABSTRACT

The present invention relates to a stent for transluminal implantation into hollow organs, in particular into blood vessels, ureters, esophagi, the colon, the duodenum, the airways or the biliary tract, comprising an at least substantially tubular body that extends along a longitudinal direction and that can be converted from a compressed state having a first cross-sectional diameter into an expanded state having an enlarged second cross-sectional diameter, wherein the stent comprises a stent body composed of a biostable material, characterized in that the stent body comprises a plurality of stent sections, preferably annular stent sections, that are in particular separate from one another, and the stent has a support structure that connects the stent sections to one another, wherein the support structure is formed from a bioresorbable material or comprises a bioresorbable material.

The present invention relates to a stent for transluminal implantation into hollow organs, in particular into blood vessels, ureters, esophagi, the colon, the duodenum, the airways or the biliary tract, comprising an at least substantially tubular body that extends along a longitudinal direction. The stent can be converted from a compressed state having a first cross-sectional diameter into an expanded state having an enlarged second cross-sectional diameter that is enlarged in comparison with the first cross-sectional diameter. The stent comprises a stent body composed of a biostable material.

Stents are used to treat pathologically altered hollow organs, for example, when the hollow organs have narrowed (stenosis). Another field of application is the treatment of aneurysms.

For the treatment, the stent is introduced in the compressed state via an insertion catheter to the position within the hollow organ to be treated where the stent is expanded by dilatation or by self-expansion to a diameter that, for example, corresponds to the diameter of the healthy hollow organ so that a supporting effect of the hollow organ, in particular of a vessel wall, is achieved.

Stents are usually intended to provide a high radial deployment force in order to have a sufficiently large supporting effect, e.g. for a blood vessel. Furthermore, the stent should be able to adapt to deformations of the hollow organ that, for example, arise through movements of the patient. For this reason, the stent should be flexible to allow deformations in the longitudinal direction.

Furthermore, it is desirable to be able to place the stent in the hollow organ in a simple manner and in a precise location.

It is therefore the underlying object of the invention to provide an improved stent that in particular takes into account the aforementioned aspects.

This object is satisfied by a stent in accordance with claim 1.

The stent in accordance with the invention is characterized in that the stent body comprises a plurality of stent sections, preferably annular stent sections, that are in particular separate from one another. Furthermore, the stent in accordance with the invention comprises a support structure that connects the annular stent sections to one another, wherein the support structure is formed from a bioresorbable material or comprises a bioresorbable material.

The bioabsorbable material is degraded or resorbed after the insertion into the hollow organ so that after a certain period of time only the stent body composed of the biostable material (permanently) remains in the hollow organ. The invention is therefore based on the recognition that the stent sections of the stent body can be connected to one another by the support structure composed of the bioresorbable material and can e.g. be fastened to one another in this manner in order to facilitate the insertion of the stent body into the hollow organ. After the dissolution or the resorption of the bioresorbable material, the connection of the stent sections by the support structure is then omitted so that the flexibility of the stent can e.g. increase along the longitudinal direction.

The support structure can therefore be a structure that remains in the body only temporarily and that is advantageous during the insertion of the stent into the hollow organ, but is not intended to permanently remain in the hollow organ.

The stent sections are preferably separate from one another. This means that the individual stent sections are not connected to one another by a biostable material of the stent body, but are e.g. only connected to one another by the support structure. Due to the separate stent sections, a particularly high flexibility along the longitudinal direction results after the omission of the support structure. Furthermore, the stent sections can be annular, wherein, for example, an equal spacing can in each case be present between the annular stent sections. Alternatively or additionally, any desired other shapes are, however, also possible, for example, with chamfered and/or irregularly shaped stent sections.

The individual stent sections are in particular arranged lined up after one another along the longitudinal direction and preferably have a common axis that extends in the longitudinal direction.

The stent generally has a substantially tubular body. This means that the length of the stent in the longitudinal direction is preferably greater (e.g. at least twice, five times, ten times, or thirty times greater) than the cross-sectional diameter of the stent. In the expanded state, the stent can have an empty, continuous lumen/volume in its interior through which e.g. a blood flow is possible. The stent can, over its total length in the longitudinal direction, have a cross-sectional diameter that is substantially constant in the expanded state. Alternatively, the cross-sectional diameter can also change over the length of the stent, for example, decrease continuously to account for a decrease in diameter of the hollow organ.

The stent can be a self-expanding stent that transfers itself from the compressed state into the expanded state without an active force effect.

Alternatively, the stent can also be non-self-expanding and balloon-expandable, for example. A balloon can then be placed in the interior of the stent, wherein the balloon is inflated under pressure, enlarges and, during this enlargement, urges and transfers the stent from the compressed state into the expanded state (“balloon dilation”).

The biostable material can, for example, be a nickel-titanium alloy (nitinol). The stent body is preferably formed from a shape-storing material (“shape memory”) that assumes the stored shape from a limit temperature onward. In addition to said nickel-titanium alloy, the stent body can also be formed as cobalt-chromium alloys, cobalt-nickel alloys or platinum-chromium alloys or comprise such alloys. Furthermore, other suitable metals and/or metal alloys and/or metal materials are also possible.

The stent can in particular be a “covered stent” that is e.g. surrounded by a fabric material. The stent can also comprise a stent graft. The stent graft is in particular an artificial vessel wall that can, for example, be used in the treatment of aneurysms. However, the stent described herein can also be a “bare stent,” i.e. a stent without a stent graft or a fabric material.

The support structure in particular comprises a plurality of separate parts, i.e. the support structure preferably does not form a single coherent structure.

The stent body can comprise a plurality of cells that are defined by bordering elements (e.g. so-called “struts”) formed by the stent body. The bordering elements can be produced by removing material when cutting the stent body from a material, in particular a tubular material, wherein the bordering elements or struts remain. The bordering elements are preferably fixedly connected to one another and in particular in one piece. Accordingly, the stent body, for example, does not comprise a braided material.

A cell can be connected to one or more other cells by one connection section of by a plurality of connection sections. A cell comprises the total recess as well as its respective bordering elements, wherein the connection sections belong to the bordering elements.

The cells of the stent body preferably form a convex polygon and in particular have a diamond shape. Alternatively, the cells can also be round, circular or elliptical. The aforementioned shapes of the cells can in particular result when the cells are placed or pressed onto a plane (the so-called unwinding). In a convex polygon, all the interior angles each have an angle of ≤180°. Such cells can also be called closed cells. Due to the shape of a convex polygon and in particular due to the diamond shape or also due to an elliptical shape, a high deployment force and thus a high supporting effect of the stent can result.

Most or all of the cells in particular have the shape of a convex polygon, a diamond shape, an annular shape, an elliptical shape, and/or a circular shape.

Alternatively or additionally, cells of the stent body can also form a concave polygon. In the concave polygon, at least one interior angle can have an angle of >180°. For example, at least some of the bordering elements of a respective cell can have a zigzag shape. Due to such zigzag cells or generally due to cells having the shape of a concave polygon, the flexibility of the stent can be further increased. Most or all of the cells can also have the shape of a concave polygon.

Furthermore, it is possible that the majority or all the cells of the stent body have the same or a similar shape. A similar shape can in particular be spoken of when the bordering elements of two cells have a maximum offset when they lie on one another that does not exceed 10%, 20% or 30% of the length of the greatest extent of the cell in one spatial direction.

At least one of the stent sections preferably comprises a row of cells that follow one another in the peripheral direction of the stent and that preferably form a closed ring running around in the peripheral direction of the stent. The ring preferably has a cross-section, which is closed in annular form and which is formed by the cells of the ring, perpendicular to the longitudinal direction of the stent. An ideal radial deployment force and supporting effect of the stent or of the stent body can be provided in the stent section by such a ring that runs around in the peripheral direction and that is preferably closed. A stent section can in principle be formed by any desired cells. An annular stent section can in particular have exactly one single row or exactly two or three rows of cells connected to one another. Due to two or three rows of cells connected to one another, the stent section is extended in the longitudinal direction, but also provides an increased deployment force.

Advantageous further developments of the invention can be seen from the description, from the drawings and from the dependent claims.

In accordance with a first embodiment, the support structure is configured to hold the stent sections in a defined relative position to one another. The support structure therefore fastens the stent sections so that their spacing and/or orientation remains/remain in the expanded and/or compressed state (at least without a greater force effect from the outside). A constant spacing and/or orientation enables an improved insertion at a very precisely determinable target position. During the resorption of the support structure in the hollow organ, some ingrowth in the stent sections usually takes place so that the stent sections then also remain at their respective positions without the support structure. However, as already mentioned, the flexibility also preferably increases along the longitudinal direction without the support structure. Alternatively or additionally, the flexibility of the stent body can generally improve, thus the longitudinal stretchability and/or compressibility can be increased and/or the torsional capacity can improve, wherein no relevant reduction of the radial deployment force, i.e. of the supporting effect, takes place.

The connection or the fastening of the stent sections by the support structure can in particular also be understood such that relative movements of the support structure and the stent body or stent sections are still possible, but that an unintentional loosening of the support structure and the stent body is prevented.

In accordance with a further embodiment, the support structure is arranged at least substantially at the outer side of the stent body. When inserted in the hollow organ, the outer side of the stent body at least regionally contacts the tissue of the hollow organ. More than 50%, more than 80%, or more than 95% of the material of the support structure can in particular lie at the outer side and/or outside the stent body. The support structure preferably does not project beyond an inner side of the stent body into the free lumen in the interior of the stent in order thus to avoid an impairment e.g. of the blood flow through the free lumen.

The parts of the support structure are therefore in particular arranged such that they are pressed against the wall of the hollow organ or at least come to lie in the region of the wall of the hollow organ so that fragments of the support structure are e.g. prevented from entering the blood stream and being flushed away during the resorption process.

In accordance with a further embodiment, the support structure comprises a plurality of rails that extend at least substantially in parallel with the longitudinal direction. The rails can preferably be fastened to connection sections of the cells of the stent body. The rails are further preferably each straight and e.g. extend at the outer side of the stent body. Due to the rails, the necessary forces for inserting and/or releasing the stent into/from a catheter of an insertion set of instruments can be reduced. Both the preparation of the insertion procedure and the release of the stent in the hollow organ are thereby simplified. The rails thus create an advantage during the insertion of the stent, then release due to the bioresorbable material and do not hinder the flexibility of the stent in the long term.

Alternatively or additionally, at least some rails can also have a spiral course along the longitudinal direction, i.e. at least partly revolve around an axis formed by the stent.

In accordance with a further embodiment, the rails are uniformly distributed in the peripheral direction of the tubular body. This means that, viewed in cross-section, the rails are arranged uniformly, for example, along the periphery of the stent body. Viewed from a central axis of the stent body, a rail can then be arranged at least every 30°, every 45° or every 60°, for example.

In accordance with a further embodiment, the rails have an equal length in the longitudinal direction. The length of the rails can, for example, correspond to the length of the stent body. The rails can also be formed shorter than the stent body and can, for example, only have 30, 40, 50, 60 or 70% of the length of the stent body in the longitudinal direction. Alternatively or additionally, at least some of the rails can also be longer than the stent body.

In accordance with a further embodiment, the rails have different positions, viewed in the longitudinal direction. The rails can therefore have different initial and/or end positions in the longitudinal direction. For example, a respective two rails can only partly overlap along the longitudinal direction. It is also possible for the rails to have a spacing from the ends of the stent body or to project beyond the ends of the stent body.

In accordance with a further embodiment, at least one of the rails comprises at least one spring element that has an increased flexibility in comparison with the flexibility of the rail in the region outside the spring element, wherein the spring element is preferably arranged between two stent sections. The spring element is further preferably arranged centrally between two stent sections. Due to the spring element, the bendability or flexibility of the stent is in particular increased in the longitudinal direction since a certain movability of the stent sections from one another can be made possible by the increased flexibility of the spring element. The spring element preferably comprises a tapered region and/or a zigzag shape and/or a meandering shape, wherein the increased flexibility of the spring element is achieved by the tapered region and/or the zigzag shape and/or the meandering shape. In particular due to the positioning between the stent sections, the stent sections are then flexibly movable with respect to one another, as explained above.

In accordance with a further embodiment, the support structure is fastened to the stent body by means of a form fit and/or a force fit. The fastening of the support structure to the stent body in particular takes place solely by means of a form fit and/or a force fit. The form fit and/or force fit preferably furthermore takes place solely with material of the stent body and the support structure, i.e. no additional material is required, for example, for a bonding, soldering, welding and the like.

Due to the connection of the support structure and the stent body by means of a form fit and/or a force fit, it is made possible to connect the biostable material to the bioresorbable material even though nitinol is, for example, practically not weldable to a bioresorbable material such as zinc. Due to the fastenings described herein of the support structure to the stent body, a reasonable use in the hollow organ is then nevertheless possible.

The connection of the support structure and the stent body by means of a form fit and/or a force fit can in particular take place such that a recess with an undercut is provided in the stent body, wherein the support structure projects into the undercut. For example, the recess with the undercut can be approximately trapezoidal (e.g. formed as an isosceles trapezoid), viewed in cross-section, wherein the trapezoid can be open at its shorter base side, whereby the undercut is produced in the region of the longer base side.

The material of the support structure can be introduced into the region of the recess with the undercut and can be heated there, e.g. to briefly liquefy it. The material of the support structure can thereby contact the stent body in the region of the undercut and can create a form fit and, if necessary, also a force fit in this manner.

In accordance with a further embodiment, for fastening the support structure to the stent body, the stent body has a fastening projection around which the support structure runs at least in part. The support structure can preferably also completely run around the fastening projection. It is thus a form-fitted connection of the stent body and the support structure. The support structure, for example, describes an arc (for a partial running around) or a loop (for a complete running around). It is thus a form-fitted connection of the support structure and the stent body.

The support structure can preferably be flattened in the region of the fastening projection in the manufacturing process of the stent in order, for example, to be planar with the inner side of the stent body and not to project into the lumen of the stent body.

In accordance with a further embodiment, the fastening projection is arranged in a recess of the stent body and/or projects from the recess. The recess can, for example, have a circular or elliptical shape in cross-section and can accordingly form an eyelet in which the fastening projection is arranged and/or from which the fastening projection projects. The fastening projection in particular arises from the inner wall of the recess and (initially) projects into the recess. When partly or completely running around the fastening projection, the support structure can then be supported against the inner wall of the recess, whereby a secure connection between the support structure and the stent body is produced.

The fastening projection can, for example, be curved and can, in particular only, partly project from the recess. The fastening projection preferably projects from the outer side of the stent body in order not to impair the lumen in the interior of the stent body.

In accordance with a further embodiment, for fastening the support structure, the stent body is hooked to the support structure, in particular by means of two fastening rings engaging into one another, wherein at least one of the fastening rings is open. The support structure can, for example, have one or more closed fastening rings, whereas the stent body comprises one or more open fastening rings. The fastening rings can then be connected to one another like chain members. Alternatively, the open fastening rings can also be provided at the support structure and the closed fastening rings can be provided at the stent body. The fastening rings are preferably ring disks, however, other shapes are also possible, for example oval, square, spoon-shaped (i.e. curved, in particular with the same radius as the stent body). The shape of the fastening rings can generally be arbitrary as long as a fastening ring of the stent body can engage into a fastening ring of the support structure.

In an open fastening ring, a slot can be provided that is wider than the thickness of the material of the closed fastening ring. However, the slot can also have a maximum width of twice, three times or five times the thickness of the material of the fastening ring.

The fastening by the fastening rings is likewise not a form-fitting connection. In general, the stent body can therefore be connected to the support structure via hooks and eyelets.

In accordance with a further embodiment, a hooking of the stent body and the support structure is achieved by means of a barb guided through an opening. The opening can in particular be a closed fastening ring, as described above. The closed fastening ring can preferably be attached to the stent body in this case. The barb can be formed from a bioresorbable material and comprises a bar having at least two rearwardly directed hook sections attached to the tip of the bar. The hook sections can define a V shape together. On the manufacture of the stent, the hook sections can be guided through the opening after one another, wherein a pulling out of the hook sections is thereafter prevented. Due to the barb at the support structure, a fastening of the support structure to the stent body can thus be achieved.

In accordance with a further embodiment, the support structure comprises a plurality of cylinder segments in the interior of the stent body. The cylinder segments preferably contact the inner wall of the stent body. The cylinder segments are produced by a cutting of a cylinder along the cylinder axis. In cross-section, the cylinder segments can have the shape of circular sectors or circular ring sections. The circular ring sections can in particular form a hollow cylinder when combined.

In accordance with a further embodiment, the cylinder segments form a cylinder or a hollow cylinder in a compressed state of the stent. On the transition into the expanded state, the cylinder segments then move away from one another and form rails, in particular inwardly disposed rails, attached to the stent body. The cylinder segments can be fastened to the stent body by means of the fastening possibilities mentioned herein.

Alternatively or in addition to an inwardly disposed hollow cylinder, it is also possible that the support structure forms an outwardly disposed hollow cylinder or an outwardly disposed tube around the stent body, in particular in the compressed state.

The inwardly disposed support structure can in particular have a different degradation behavior than the outwardly disposed support structure, for example, the inwardly disposed support structure can be more quickly biodegradable than the outwardly disposed support structure. Thus, the inwardly disposed support structure can e.g. be fully degradable within minutes or hours, whereas the outwardly disposed support structure can take days or weeks for the complete degradation.

In accordance with a further embodiment, the support structure comprises a plurality of recesses and/or depressions in which stent sections of the stent body come to lie. In this case, the support structure can in particular be outwardly disposed, i.e. arranged at the outer side of the stent body. The recesses can also be referred to as positioning recesses. The recesses/depressions can be adapted for the stent sections so that thickened portions of the support structure are then present between the stent sections. The recesses or depressions hold the stent sections and thus the stent body in their position. Due to the recesses or depressions, a precise positioning of the individual stent sections can thus likewise be achieved during the insertion into the hollow organ.

In accordance with a further embodiment, at least two stent sections of the stent body are connected to one another by connection elements formed from the material of the stent body, wherein parts of the support structure are attached to the connection elements to reinforce the connection elements. In this embodiment, the stent sections are not formed separately from one another at least in part. To reinforce the connection elements by means of the bioresorbable material, the connection elements can, for example, be coated by the bioresorbable material. Alternatively or additionally, the connection elements can e.g. have a groove or another depression into which the bioresorbable material of the support structure is inserted. On the insertion of the stent into the hollow organ, the bioresorbable material then causes an increased stiffness of the connection elements, which in turn results in good positioning properties. After the resorption of the bioresorbable material, the connection elements are then more flexible due to the omission of the additional material, whereby the flexibility of the stent then advantageously increases.

In accordance with a further embodiment, the connection elements at least regionally have a thinner and/or tapered material. The material is in particular thinner and/or tapered compared to the stent sections of the stent body. The thinner and/or tapered material in this respect only refers to the biostable material of the stent body. Thinner, for example, means less area and/or material in cross-section than in cross-section e.g. through a strut in a stent section. Together with the material of the support structure, the connection element can be just as thick or even thicker than the biostable material in the region of the stent sections.

In accordance with a further embodiment, the support structure presses against the stent body in at least one fastening recess of the stent body to fasten the support structure to the stent body by means of a force fit. The support structure can preferably be regionally pressed with the stent body. The fastening recess can, for example, be a hole or a passage hole in the stent body into which the bioresorbable material of the support structure is pressed. The pressing in can, for example, take place by a pressing mandrel, as explained in more detail below. As a result, the bioresorbable material of the support structure can press against the inner wall of the fastening recess at all sides, wherein a recess in the bioresorbable material likewise centrally remains in the fastening recess.

In accordance with a further embodiment, the stent body is formed from a biostable material that stores a shape and that assumes the stored shape from a limit temperature onward, wherein the biostable material, for example, is or comprises nitinol, as already discussed above.

Furthermore, the stent can comprise a plurality of X-ray markers, in particular composed of tantalum, for a simpler positioning under X-ray observation. The X-ray markers can, for example, be fastened to the ends of the stent body and/or of the support structure, viewed in the longitudinal direction. X-ray markers at the support structure can in particular be formed by thickened material portions of the bioresorbable material. At least one X-ray marker, in particular in the form of an eyelet, preferably extends away from at least one end of the stent body in the longitudinal direction, wherein the X-ray marker has an asymmetrical shape. A marker can be a section of the stent body that has an increased radiopacity, i.e. is particularly easily visible in an X-ray. The marker can in particular be an eyelet that is, for example, filled with or covered by the aforesaid tantalum.

In accordance with a further embodiment, the bioresorbable material comprises zinc. The bioresorbable material preferably consists of zinc and silver or comprises zinc and silver. The bioresorbable material in particular includes 90.0 to 99.95 mass % zinc and 0.05 to 10.0 mass % silver. Such a bioresorbable material can, for example, be degraded by the body in the bloodstream within a few weeks.

Alternatively, the bioresorbable material can also comprise or consist of a polymeric material, for example, poly-lactic acid (PLA) or poly-L-lactic acid (PLLA). The bioresorbable material can also comprise a magnesium alloy or consist of a magnesium alloy. Due to the above-described use of zinc or a zinc alloy, the radiopacity of the stent can, however, be increased compared to PLA, PLLA or magnesium alloys. The bioresorbable material can also be referred to as a biodegradable material.

Combinations of different bioresorbable materials are also possible. The support structure can likewise comprise one or more different bioresorbable materials and one or more radiopaque materials.

The stent body and/or the support structure can in particular also have an active agent added to it or be coated by an active agent. Such an active agent can have an anti-proliferative effect to prevent a tissue overgrowth of the stent. For example, anti-proliferatives of the limus group, statins, P2Y12 antagonists or thrombin antagonists can be used as the active agent.

A further object of the invention is a stent system comprising a stent of the type described herein and a catheter in which the stent is received or can be received in a compressed state. The stent is preferably surrounded by a cover in the catheter. On the insertion of the stent into the catheter, i.e. on the preparation of the stent system, the force required for the insertion into the catheter can in particular be reduced by rails of the support structure. The force required for the release from the catheter or the cover can likewise be reduced, which enables an easier handling of the stent system. The catheter can preferably have depressions at an inner wall, in which depressions the rails of the stent or, in general, the support structure comes to lie to be able to position the stent in the catheter in a simple manner.

The catheter can be part of a so-called insertion set of instruments, wherein the insertion set of instruments in particular comprises a handling means for moving and releasing the stent in the hollow organ.

The catheter can in particular be configured to pull the stent sections closer together during the release in order to simplify the release and in particular to reduce the force required for the release. Alternatively or additionally, it is possible that the catheter is configured to cause a rotation of the stent and in particular of the stent body during the release of the stent so that the cells of the stent engage into one another in the manner of toothed wheels, i.e. that in each case a tip of a cell projects into a bulge between two cells (“peak to valley”). In this way, a more continuous surface of the stent can be produced by which a rotation of the catheter by a few degrees alternately to the right and the left can be avoided (which can otherwise be necessary when the tips of cells of adjacent stent sections are precisely oriented toward one another).

Alternatively or additionally, the catheter can also be configured to design a spacing of the stent sections from one another in a settable manner during the release, e.g. in three steps (tight, normal, wide). Such a spacing change can be made possible by the elasticity of the support structure.

The contraction and/or rotation of the stent sections can e.g. take place by holding the stent, in particular at the ends of the stent, by means of two separate holding mechanisms of the catheter, wherein the two holding mechanisms are moved and/or rotated with respect to one another.

A further object of the invention is a method of manufacturing a stent comprising an at least substantially tubular body that extends along a longitudinal direction and that is convertible from a compressed state having a first cross-sectional diameter into an expanded state having an enlarged second cross-sectional diameter, wherein the stent comprises a stent body composed of a biostable material, wherein the stent body comprises a plurality of stent sections, preferably annular stent sections, that are in particular separate from one another, and the stent has a support structure that connects the annular stent sections to one another, wherein the support structure is formed from a bioresorbable material or comprises a bioresorbable material, wherein, in the method, a force is applied to the support structure already contacting the stent body in order to bring about a deformation of the support structure. The deformation can, for example, be a flattening of a loop of the support structure to push parts of the support structure projecting into the interior space out of the interior space.

In accordance with an embodiment of the method, a pressing of the support structure with the stent body takes place on the exertion of the force in order to fasten the support structure to the stent body by means of a force fit. The pressing preferably takes place in a fastening recess.

Generally speaking, the support structure can be pressed to the stent body at one or more points, wherein the support structure is pushed over material of the stent body and then pressed, for example, in a pressed region.

In accordance with a further embodiment of the method, an inner tube is inserted as a counter-bearing into the stent body during the pressing. The pressing can, for example, take place by a pressing mandrel having a conical shape. During the pressing, an inner tube can be inserted as a counter-bearing in the interior of the stent body so that the stent body does not collapse due to the pressure of the pressing mandrel. The inner tube can have a recess through which the pressing mandrel can be pushed into the interior of the inner tube during the pressing. The support structure can be pressed in an annular shape and/or at all sides against walls of the fastening recess by the pressing mandrel, wherein a region without material of the support structure is in particular centrally produced in the fastening recess. The region without material is produced after the removal of the pressing mandrel where the pressing mandrel was located during the pressing.

Due to the pressing of the support structure and the stent body, a durable connection can be provided between the biostable material and the bioresorbable material.

The statements made about the stent in accordance with the invention apply accordingly to the stent system in accordance with the invention and the method in accordance with the invention. This in particular applies with respect to advantages and embodiments. It is furthermore understood that all the embodiments mentioned herein can be combined with one another, unless explicitly stated otherwise.

The invention will be described purely by way of example with reference to the drawings in the following. There are shown:

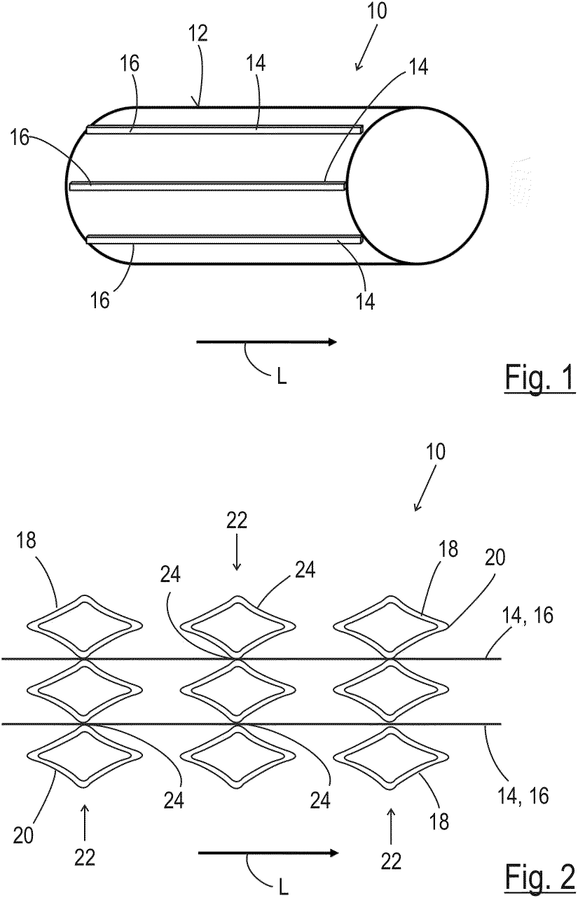

FIG. 1 schematically, a stent with a stent body and a support structure in accordance with a first embodiment;

FIG. 2 schematically, the cells of a stent and rails of the support structure in accordance with a second embodiment;

FIG. 3 schematically, the cells of a stent and rails of the support structure in accordance with a third embodiment;

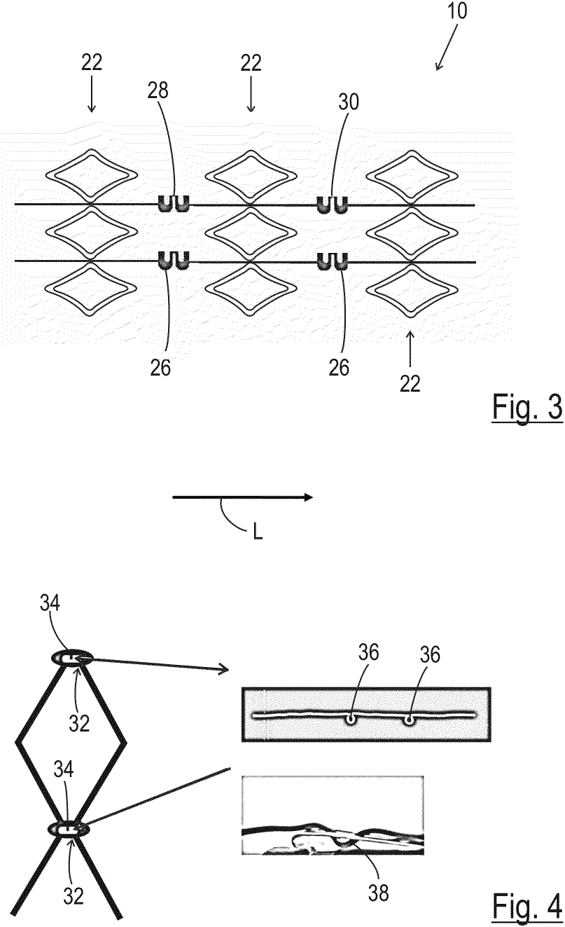

FIG. 4 schematically, the connection of the support structure to the stent body in accordance with a fourth embodiment;

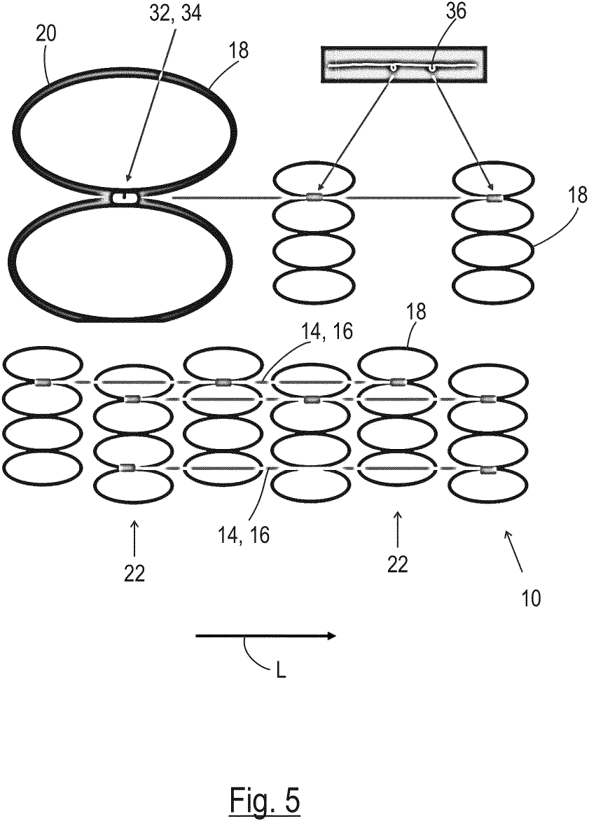

FIG. 5 schematically, the fastening of the support structure to cells of the stent body in accordance with a fifth embodiment;

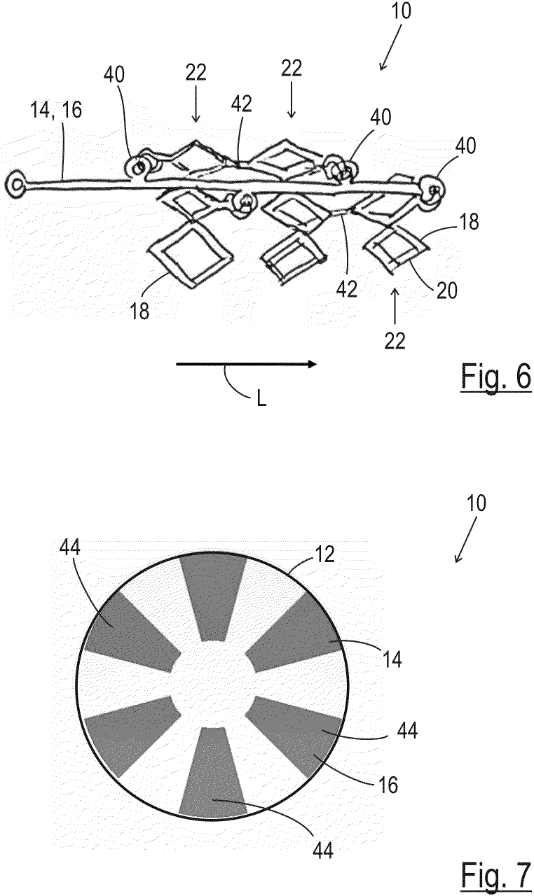

FIG. 6 schematically, the connection of the support structure to the stent body in accordance with a sixth embodiment;

FIG. 7 a stent body and a support structure that is configured as a hollow cylinder divided into cylinder segments in the interior of the stent body (seventh embodiment);

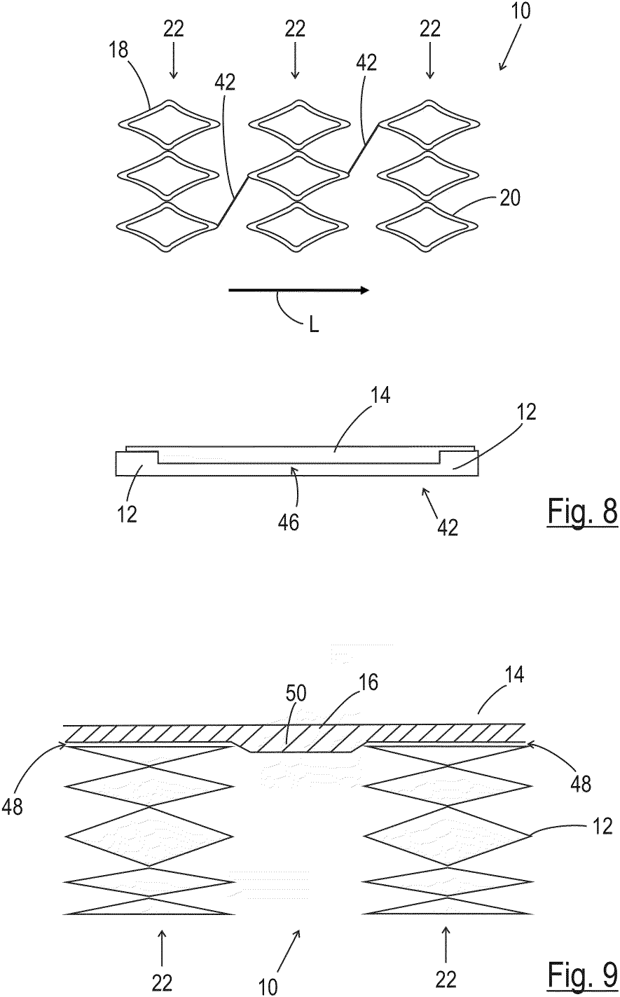

FIG. 8 schematically, connection elements between stent sections that are reinforced by a bioresorbable material (eighth embodiment); and

FIG. 9 a support structure in accordance with a ninth embodiment, wherein the support structure comprises depressions in which stent sections come to lie.

FIG. 1 shows a stent 10 of a first embodiment in the expanded state. The stent 10 has a tubular shape and extends along a longitudinal direction L. The stent comprises a tubular stent body 12, wherein the stent body 12 is connected to a support structure 14. In FIG. 1, the support structure 14 is shown in the form of a plurality of rails 16.

The stent body 12 is formed from a biostable material, for example, nitinol. The support structure 14, in contrasts, consists of a bioresorbable material, for example, a zinc alloy.

For the further embodiments, essentially only the differences from the embodiment of FIG. 1 are explained.

FIG. 2 shows a second embodiment of the stent 10. FIG. 2 in this respect shows a section of an unwinding of the stent 10. The stent body 12 is formed from cells 18. The cells 18 each have a diamond shape and are formed from strut-like bordering elements 20.

The stent 10 can each have additional cells 18 that are not shown in the Figures.

The cells 18 shown at the top in FIG. 2 are each connected to the cells 18 shown at the bottom in FIG. 2 to create the tubular shape of the stent body 12 in this manner. The cells 18 of the stent 10 each form annular stent sections 22 that are separate from one another. Each stent section 22 has a row of cells 18 that are connected to one another in the peripheral direction. The cells 18 of a stent section 22 are each connected to the cells 18 adjacent in the peripheral direction via two connection sections 24.

The rails 16 are each coupled to the stent body 12 in the region of the connection sections 24. The rails 16 are elongated and straight and extend over the total length of the stent body 12. The individual stent sections 22 themselves are only connected to one another via the rails 16 of the support structure 14.

FIG. 3 shows a third embodiment of the stent 10. The third embodiment differs from the embodiment in accordance with FIG. 2 in that the rails 16 each have a spring element 26 between two stent sections 22. The spring element 26 comprises a tapering 28 that is part of a meandering structure 30. The spring elements 26 allow a certain movability of the stent sections 22 with respect to one another.

FIG. 4 shows a fourth embodiment, wherein one possibility of connecting the support structure 14 to the stent body 12 is shown. FIG. 4 shows recesses 32 formed in connection sections 24, wherein a fastening projection 34 projects into the recesses 32. Loops 36 or arcs 38 of the support structure 14 can then be placed around the fastening projections 34 to fasten the support structure 14 to the stent body 12.

The fifth embodiment shown in FIG. 5 likewise comprises fastening projections 34 around which loops 36 of the support structure 14 are placed. In contrast to the embodiment of FIG. 4, the fifth embodiment comprises elliptical cells 18. Furthermore, the fifth embodiment shows rails 16 that are each only fastened to each second stent section 22 (viewed in the longitudinal direction L). Furthermore, the rails 16 are formed shorter than the length of the stent body 12.

FIG. 6 shows a sixth embodiment of the stent 10. In the sixth embodiment, the support structure 14 is connected to the stent body 12 by means of fastening rings 40. Both the stent body 12 and the support structure 14 have fastening rings 40 in this respect. The fastening rings 14 of the stent body 12 can in this respect be open and have a slot (not shown) through which the fastening ring 40 of the support structure 14 can engage in the fastening ring 40 of the stent body 12. Accordingly, the fastening rings 40 of the support structure 14 can be closed fastening rings 40.

In the embodiment in accordance with FIG. 6, the individual stent sections 22 can also be connected to one another by material of the stent body 12, more precisely by connection elements 42. The connection elements 42 each extend from an end of a diamond-shaped cell 18 of a stent section 22 up to an end of a cell 18 of a directly adjacent stent section 22.

FIG. 7 shows a seventh embodiment of a stent 10 in the expanded state. FIG. 7 in this respect shows a view in the direction of the longitudinal axis L. Within the stent body 12, the support structure 14 comprises a plurality of cylinder segments 44 that form a hollow cylinder in the compressed state. The cylinder segments 44 form inwardly disposed rails 16 within the stent body 12.

FIG. 8 shows an eighth embodiment of the stent 10 in which the stent sections 22 are connected by connection elements 42. In the upper region of FIG. 8, a part of an unwinding of the stent 10 is shown for this purpose (top view). In the lower part of FIG. 8, a side view of a connection element 42 is shown. The connection element 42 is formed from a biostable material of the stent body 12 and is reinforced by a bioresorbable material of the support structure 14. The material of the support structure 14 serving for the reinforcement is received in a receiver 46 of the connection element 42, wherein the receiver 46 can e.g. be formed by a recess in the connection element 42.

FIG. 9 shows a ninth embodiment of the stent 10, wherein a schematic side view of the stent 10 is shown. The stent 10 again comprises a plurality of stent sections 22 that are connected to a support structure 14, wherein a rail 16 of the support structure 14 is shown in FIG. 9. The rail 16 comprises a plurality of positioning recesses 48 in which the stent sections 22 come to lie and are held in predefined positions relative to one another in this manner. A thickened portion 50 of the rail 16 is provided between the positioning recesses 48.

As stated above, the different embodiments can be combined with one another. For example, the positioning recesses 48 can also be integrated in the rails 16 of the preceding embodiments. It is likewise possible to combine different connection methods between the stent body 12 and the support structure 14 with one another.

It is a common feature of all the embodiments that the support structure 14 causes an increased stability of the stent 10 during the insertion into a hollow organ. The support structure 14 is only temporarily present after the insertion and is resorbed, wherein the advantage of an increased flexibility of the stent 10 is given after the resorption.

Reference numeral list

| | |
|---|---|
| 10 | stent |
| 12 | stent body |
| 14 | support structure |
| 16 | rail |
| 18 | cell |
| 20 | bordering element |
| 22 | stent section |
| 24 | connection section |
| 26 | spring element |
| 28 | tapering |
| 30 | meandering structure |
| 32 | recess |
| 34 | fastening projection |
| 36 | loop |
| 38 | arc |
| 40 | fastening ring |
| 42 | connection element |
| 44 | cylinder segment |
| 46 | receiver |
| 48 | positioning recess |
| 50 | thickened portion |

1. A stent for transluminal implantation into hollow organs, the stent comprising an at least substantially tubular body that extends along a longitudinal direction and that can be converted from a compressed state having a first cross-sectional diameter into an expanded state having an enlarged second cross-sectional diameter, wherein the stent comprises a stent body composed of a biostable material,
wherein
the stent body comprises a plurality of stent sections and
the stent has a support structure that connects the stent sections to one another, wherein the support structure is formed from a bioresorbable material or comprises a bioresorbable material.

2. The stent in accordance with claim 1,
wherein the support structure is configured to hold the stent sections in a defined relative position to one another,
and/or
wherein the support structure is arranged at least substantially at the outer side of the stent body.

3. The stent in accordance with claim 1,
wherein the support structure comprises a plurality of rails that extend at least substantially in parallel with the longitudinal direction.

4. The stent in accordance with at least claim 3, wherein at least one of the rails comprises at least one spring element that has an increased flexibility in comparison with the flexibility of the rail in the region outside the spring element.

5. The stent in accordance with claim 1,
wherein the support structure is fastened to the stent body by means of a form fit and/or a force fit.

6. The stent in accordance with claim 1,
wherein, for fastening the support structure to the stent body, the stent body has a fastening projection around which the support structure runs at least in part.

7. The stent in accordance with claim 1,
wherein, for fastening the support structure, the stent body is hooked to the support structure, by means of two fastening rings, and wherein at least one of the fastening rings is open.

8. The stent in accordance with claim 1,
wherein the support structure comprises a plurality of rails that extend at least substantially in parallel with the longitudinal direction,
wherein at least one of the rails comprises at least one spring element that has an increased flexibility in comparison with the flexibility of the rail in the region outside the spring element and
wherein the support structure comprises a plurality of cylinder segments in the interior of the stent body.

9. The stent in accordance with claim 1,
wherein the support structure comprises a plurality of recesses and/or depressions in which stent sections of the stent body come to lie.

10. The stent in accordance with claim 1,
wherein an inwardly disposed support structure has a different degradation behavior than an outwardly disposed support structure.

11. The stent in accordance with claim 1,
wherein at least two stent sections of the stent body are connected to one another by connection elements formed from the material of the stent body,
wherein parts of the support structure are attached to the connection elements to reinforce the connection elements.

12. The stent in accordance with claim 1,
wherein the support structure presses against the stent body in at least one fastening recess of the stent body to fasten the support structure to the stent body by means of a force fit.

13. A stent system comprising a stent for transluminal implantation into hollow organs, the stent comprising an at least substantially tubular body that extends along a longitudinal direction and that can be converted from a compressed state having a first cross-sectional diameter into an expanded state having an enlarged second cross-sectional diameter,
wherein the stent comprises a stent body composed of a biostable material,
wherein the stent body comprises a plurality of stent sections, and
the stent has a support structure that connects the stent sections to one another, wherein
the support structure is formed from a bioresorbable material or comprises a bioresorbable material and a catheter in which the stent is received or can be received in a compressed state.

14. The stent system in accordance with claim 13, wherein the catheter is configured to set or to change a spacing of the stent sections from one another during the release and/or to rotate the stent sections with respect to one another during the release.

15. A method of manufacturing a stent comprising an at least substantially tubular body that extends along a longitudinal direction and that is convertible from a compressed state having a first cross-sectional diameter into an expanded state having an enlarged second cross-sectional diameter,
wherein the stent comprises a stent body composed of a biostable material,
wherein the stent body comprises a plurality of stent sections,
and
the stent has a support structure that connects the annular stent sections to one another, wherein the support structure is formed from a bioresorbable material or comprises a bioresorbable material,
wherein, in the method, a force is applied to the support structure already contacting the stent body in order to bring about a deformation of the support structure.

16. The stent in accordance with claim 1,
wherein the stent sections are annular stent sections.

17. The stent in accordance with claim 1,
wherein the stent sections are separate from one another.

18. The stent in accordance with claim 3,
wherein the rails are uniformly distributed in the peripheral direction of the tubular body and/or have an equal length in the longitudinal direction and/or have different positions in the longitudinal direction.

19. The stent in accordance with at least claim 4,
wherein the spring element is arranged between two annular stent sections.

20. The stent in accordance with at least claim 19,
wherein the spring element is arranged centrally between two annular stent sections.

21. The stent in accordance with claim 6,
wherein the fastening projection is in a recess of the stent body and/or projects from the recess.

22. The stent in accordance with claim 7,
wherein the two fastening rings engage into one another.

23. The stent in accordance with at least claim 7,
wherein a hooking of the stent body and the support structure is achieved by means of a barb guided through an opening.

24. The stent in accordance with claim 8,
wherein the cylinder segments form a cylinder or a hollow cylinder in a compressed state of the stent.

25. The stent in accordance with claim 10,
wherein the inwardly disposed support structure is more quickly biodegradable than the outwardly disposed support structure.

26. The stent system in accordance with claim 13,
wherein the catheter has depressions at an inner wall, in which depressions the support structure of the stent comes to lie.
